(19) United States
(12) Patent Application Publication
(10) Pub. No.: US 2008/0171687 A1
(43) Pub. Date: Jul. 17, 2008

(54) Compositions And Methods For The Preparation And Administration Of Poorly Water Soluble Drugs

(75) Inventors: Neil P. Desai, Los Angeles, CA (US); Chunlin Tao, Los Angeles, CA (US); Andrew Yang, Rosemead, CA (US); Bridget Beal-Grim, Torrance, CA (US); Tapas De, Los Angeles, CA (US); Patrick Soon-Shiong, Los Angeles, CA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Abraxis Bioscience, Inc., Los Angeles, CA (US)

(21) Appl. No.: 11/575,031

(22) PCT Filed: Sep. 16, 2005
(86) PCT No.: PCT/US2005/033396

§ 371 Date: Oct. 19, 2007

Related U.S. Application Data
(60) Provisional application No. 60/610,407, filed on Sep. 16, 2004.

(51) Int. Cl. A61K 38/02; A61K 31/351; A61K 31/222; A61K 31/427

(52) U.S. Cl. 514/2; 514/459; 514/365; 514/551

(57) ABSTRACT

Sterile, stable pharmaceutical formulations of poorly water-soluble drugs dissolved in dimethyl isosorbide, a water-miscible solvent, as well as methods for their preparation and administration.

### FIELD OF THE INVENTION

The present invention relates to drug delivery systems for poorly water soluble drugs suitable for parenteral and other routes of administration, as well as methods for their preparation and administration.

### BACKGROUND OF THE INVENTION

There is an ever increasing number of pharmaceutical drugs being formulated that are poorly soluble or insoluble in aqueous solutions. Such drugs provide challenges to delivering them in an injectable form such as through parenteral administration. A well-designed formulation must, at a minimum, be capable of presenting a therapeutically effective amount of the poorly soluble drug to the desired absorption site, in an absorbable form. Even this minimal functionality is difficult to achieve when delivery of the poorly soluble drug requires interaction with aqueous physiological environments, such as gastric fluids and intestinal fluids. Pharmaceutical compositions for delivery of poorly soluble drugs must carry the drug through the aqueous environment, while maintaining the drug in an absorbable form, while avoiding the use of physiologically harmful solvents or excipients.

A number of approaches to formulating poorly soluble drugs for oral or parenteral delivery are known. Such approaches include, for example, formulations in which the poorly soluble drug is present in an oil-in-water emulsion, a microemulsion, or a solution of micelles, liposomes, or other multi-lamellar carrier particles. While such approaches may be appropriate for some ionizable as well as non-ionizable hydrophobic therapeutic agents, they fail to take advantage of the unique acid-base chemical properties, and associated solubility properties, of ionizable compounds.

In particular, unlike non-ionizable poorly soluble drugs, ionizable poorly soluble drugs can be rendered soluble in aqueous solution if the pH of the solution is adjusted to ionize the therapeutic agent. Such an approach is well known in the art. For example, U.S. Pat. No. 5,773,029 is directed to a pharmaceutical composition of an acidic drug, wherein the solubility of the acidic drug is enhanced by simultaneous salt formation with an organic or inorganic base and complexation with a cyclodextrin. The resultant drug/cyclodextrin/base complexes reportedly are readily soluble in water in high concentrations.

U.S. Pat. No. 5,360,615 discloses a pharmaceutical carrier system for an acidic, basic or amphoteric pharmaceutical agent in which the pharmaceutical agent is partially ionized by an acid or base in a polyethylene glycol-based solvent system. The pharmaceutical agent reportedly shows enhanced solubility in the partially ionized form. The reference also discloses that addition of glycerin, propylene glycol and/or polyvinylpyrrolidone further enhances the solubility of the pharmaceutical agent in the polyethylene glycol base. However, the invention is limited to polyethylene glycol-based solvent systems and a narrow range of ionizing agent concentration, and there is no disclosure of other solvent systems. Thus, its utility is severely limited.

Similarly, U.S. Pat. No. 5,376,688 discloses a pharmaceutical solution of an acidic, basic or amphoteric pharmaceutical agent. The solution includes a pharmaceutical agent, an ionizing species, and a solvent system. The solvent system can be diethylene glycol monoethyl ether, glycerol caprylate/caprate, polyglycerol oleate, alpha-hydro-w-hydroxypoly(oxyethylene)-poly(oxypropylene)-poly(oxyethylene ) block copolymers, or mixtures of those components. The solvent system can also be a mixture of polyethylene glycol and a polyoxyethylene sorbitan ester. Optional components include water, glycerin, propylene glycol, and polyvinylpyrrolidone. However, the invention is limited to these particular compounds and a narrow range of ionizing agent concentration, rendering its utility severely limited. Moreover, some of the solvent system components show poor or questionable biocompatibility, and thus would be impractical for drug delivery to a patient.

A further problem with conventional approaches to solubilizing ionizable poorly soluble drugs is the difficulty in maintaining the solubilized therapeutic agent in solubilized form. Thus, for example, while ionizing an acidic therapeutic agent with a base may increase its solubility, the therapeutic agent is prone to precipitation in the gastrointestinal tract due to the acidic pH conditions encountered upon administration to a patient, and the approximately 10 to 100-fold dilution expected in gastrointestinal or intestinal fluids. This precipitation is particularly disadvantageous, since the precipitated therapeutic agent is essentially unavailable for absorption, leading to difficulties in controlling dosages, and a need to administer large doses of the therapeutic agent to ensure that a therapeutically effective amount reaches the absorption site in a bioavailable form. Such difficulties necessarily result in increased costs, and compromised patient safety and therapeutic effectiveness.

Drugs that are insoluble in water can have significant benefits when formulated as a stable suspension of sub-micron particles. Accurate control of particle size is essential for safe and efficacious use of these formulations. Particles must be less than seven microns in diameter to safely pass through capillaries without causing emboli (Allen et al., 1987; Davis and Taube, 1978; Schroeder et al., 1978; Yokel et al., 1981).

One approach to delivering an insoluble drug is disclosed in U.S. Pat. No. 2,745,785. This patent discloses a method for preparing crystals of penicillin G suitable for parenteral administration. The method includes the step of recrystallizing the penicillin G from a formamide solution by adding water to reduce the solubility of the penicillin G. The '785 patent further provides that the penicillin G particles can be coated with wetting agents such as lecithin, or emulsifiers, surface-active and defoaming agents, or partial higher fatty acid esters of sorbitan or polyoxyalkyklene derivatives thereof, or aryl alkyl polyether alcohols or salts thereof. The '785 patent further discloses micronizing the penicillin G with an air blast under pressure to form crystals ranging from about 5 to 20 microns.

Another approach is disclosed in U.S. Pat. No. 5,118,528, which discloses a process for preparing nanoparticles. The process includes the steps of: (1) preparing a liquid phase of a substance in a solvent or a mixture of solvents to which may be added one or more surfactants, (2) preparing a second liquid phase of a non-solvent or a mixture of non-solvents, the non-solvent is miscible with the solvent or mixture of solvents for the substance, (3) adding together the solutions of (1) and (2) with stirring, and (4) removing of unwanted solvents to produce a colloidal suspension of nanoparticles. The '528 patent discloses that it produces particles of the substance smaller than 500 nm without the supply of energy. In particular, the '528 patent states that it is undesirable to use high energy equipment such as sonicators and homogenizers.

U.S. Pat. No. 4,826,689 discloses a method for making uniformly sized particles from water-insoluble drugs or other organic compounds. First, a suitable solid organic compound is dissolved in an organic solvent, and the solution can be diluted with a non-solvent. Then, an aqueous precipitating liquid is infused, precipitating non-aggregated particles with substantially uniform mean diameter. The particles are then separated from the organic solvent. Depending on the organic compound and the desired particle size, the parameters of temperature, ratio of non-solvent to organic solvent, infusion rate, stir rate, and volume can be varied according to the invention. The '689 patent discloses the formation of a drug in a metastable state which is thermodynamically unstable and which eventually converts to a more stable crystalline state. The '689 patent further discloses trapping the drug in a metastable state in which the free energy lies between that of the starting drug solution and the stable crystalline form. The '689 patent also discloses utilizing crystallization inhibitors (e.g., polyvinylpyrrolidinone) and surface-active agents (e.g., poly(oxyethylene)-co-oxypropylene) to render the precipitate stable enough to be isolated by centrifugation, membrane filtration or reverse osmosis.

U.S. Pat. Nos. 5,091,188, 5,091,187 and 4,725,442 disclose (a) either coating small drug particles with natural or synthetic phospholipids or (b) dissolving the drug in a suitable lipophilic carrier and forming an emulsion stabilized with natural or semisynthetic phospholipids. One of the disadvantages of these approaches is their reliance on the quality of the raw material of the drug, and that they do not disclose the steps of changing the morphology of the raw material to render the material in a friable, more easily processed form.

Another approach to providing insoluble drugs for parenteral delivery is disclosed in U.S. Pat. No. 5,145,684. The '684 patent discloses the wet milling of an insoluble drug in the presence of a surface modifier to provide a drug particle having an average effective particle size of less than 400 nm. The '684 patent discloses the adsorbence of the surface modifier on the surface of the drug particle in an amount sufficient to prevent agglomeration of the individual drug particles into larger particles.

Yet another attempt to provide insoluble drugs for parenteral delivery is disclosed in U.S. Pat. No. 5,922,355. The '355 patent discloses providing submicron sized particles of insoluble drugs using a combination of surface modifiers and a phospholipid followed by particle size reduction using techniques such as sonication, homogenization, milling, microfluidization, precipitation or recrystallization. There is no disclosure in the '355 patent of changing process conditions to make crystals in a more friable form.

U.S. Pat. No. 5,780,062 discloses a method of preparing small particles of insoluble drugs by (1) dissolving the drug in a water-miscible first solvent; (2) preparing a second solution of a polymer and an amphiphile in an aqueous second solvent in which the drug is substantially insoluble whereby a polymer/amphiphile complex is formed and (3) mixing the solutions from the first and second steps to precipitate an aggregate of the drug and polymer/amphiphile complex.

U.S. Pat. No. 5,858,410 discloses a pharmaceutical nanosuspension suitable for parenteral administration. The '410 patent discloses subjecting at least one solid therapeutically active compound dispersed in a solvent to high pressure homogenization in a piston-gap homogenizer to form particles having an average diameter, determined by photon correlation spectroscopy (PCS) of 10 nm to 1000 nm, the proportion of particles larger than 5 gm in the total population being less than 0.1% (number distribution determined with a Coulter counter), without prior conversion into a melt, wherein the active compound is solid at room temperature and is insoluble, only sparingly soluble or moderately soluble in water, aqueous media and/or organic solvents. The examples in the '410 patent disclose jet milling prior to homogenization.

U.S. Pat. No. 4,997,454 discloses a method for making uniformly sized particles from solid compounds. The method of the '454 patent includes the steps of dissolving the solid compound in a suitable solvent followed by infusing precipitating liquid, thereby precipitating non-aggregated particles having a substantially uniform mean diameter. The particles are then separated from the solvent. The '454 patent discourages forming particles in a crystalline state because during the precipitating procedure the crystal can dissolve and recrystallize, thereby broadening the particle size distribution range. The '454 patent encourages during the precipitating procedure to trap the particles in a metastable particle state.

U.S. Pat. No. 5,605,785 discloses a process for forming nanoamorphous dispersions of photographically useful compounds. The process of forming nanoamorphous dispersions includes any known process of emulsification that produces a disperse phase having amorphous particulates.

Thus, there is a need for versatile and effective pharmaceutical compositions that overcome these and other deficiencies of the prior art.

Dimethyl isosorbide (DMI) is a substance having the following chemical formula:

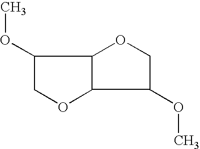

As is generally known, it is a substance with good dissolving power for organic compounds (H. P. Fiedler, Lexikon der Hilfsstoffe fur Pharmazie, Kosmetik und angrenzende Gebiete >Dictionary of Adjuvants for Pharmaceutics, Cosmetics and Related Fields, Editio Cantor Aulendorf, 1989). It has also been shown to be a good solvent, even when used without dilution with water, for use as a vehicle for intravenous and intra-arterial administration, due to its low hemolytic activity (Mottu, et al., 2001, PDA Journal of Pharmaceutical Science and Technology, 55(1):16-23). Studies have shown DMI's usefulness as an embolic liquid for the treatment of cerebral aneurysms or arteriovenous malformations (Mottu, et al., 2002, Biomaterials, 23:121-131). DMI is used in, for example, U.S. Pat. No. 4,082,881 to keep high concentrations of organic pharmaceutical substances dissolved in various topical preparations, but not in transdermal systems. In U.S. Pat. No. 4,814,173, dimethyl isosorbide is used as a solvent for a sedative, tranquilizer, antihistamine, a cognition activator, antihypertensive, analgesic, antiarrhythmic, cardiotonic, and bronchodilator, and has been used as a solvent carrier for metaxalone, methocarbamol, meprobamate, and 1-ethylcarbamoyl-3-(3-trifluoromethylphenyl)-pyrrolidine muscle relaxing drugs, e.g., in U.S. Pat. No. 3,699,230, and in U.S. Pat. No. 4,082,881 for steroids (i.e., 21 -chloro-9.alpha.-fluoro-.DELTA..sup.4 -pregnene-11.beta.,16.alpha.,17.alpha.-triol-3,20-dione and its 16,17-acetonide; 21-chloro-9-fluoro-2′,3′-dihydro-11.alpha.-hydroxy-5′-phenylpregna-1,4-die no[16.alpha., 17-b] [1,4]-dioxin-3,20-dione:acetone solvate and dichloromethane solvate (1:1); 9.alpha.-fluoro-11.beta.,16.alpha., 17,21-tetrahydroxy-pregna-1,4-diene-3,2 0-dione 16,17-acetonide, 21-(acetyloxy)-9-fluoro-1′,2′,3′,4′-tetrahydro-11.beta.-hydroxypregna-1,4-dieno[16.alpha.,17-b]naphthalene-3,20-dione, progesterone, and .DELTA.′-testololactone or a member selected from the group consisting of econazole or salts thereof, nystatin, neomycin, miconazole, gramicidin, halcinonide, triamcinolone acetonide, griseofulvin or mixtures thereof ) when used in the form of an ointment, cream, lotion or parenteral liquid, such as eye drops, etc. U.S. Pat. No. 6,071,974 discloses an injectable formulation of 2,6-diisopropylphenol which may be obtained by dissolving 2,6-diisopropylphenol in an isosorbide type solvent, e.g., dimethyl isosorbide.

It has now been found that dimethyl isosorbide is able to solubilize new classes of drugs having relatively low solubility in water, and is suitable for the delivery of these classes of drugs, thus overcoming the deficiencies in the prior art.

### SUMMARY OF THE INVENTION

The invention provides a formulation for parenteral administration to a mammal comprising DMI and a substantially water-insoluble pharmaceutically active agent selected from the group consisting of an ansamycin-derived antineoplastic agent, an epithilone, discodermolide, a discodermolide analog, actinomycin, an actinomycin analog, and combinations thereof, as well as methods for the preparation of such formulations and their administration to mammals, particularly humans.

These and other advantages of the present invention will become apparent from the subsequent detailed description of the preferred embodiments of the invention and the appended claims.

### DETAILED DESCRIPTION OF THE PREFERRED EMBODIMENTS

The present invention provides formulations for administration, preferably parenteral administration, to a mammal. These formulations comprise a water-miscible non-aqueous solvent, preferably dimethyl isosorbide (DMI), and a substantially water-insoluble pharmaceutically active agent selected from the group consisting of an ansamycin-derived antineoplastic agent, discodermolide, a discodermolide analog, an epithilone, actinomycin, an actinomycin analog, and combinations thereof. The invention further provides methods for the preparation of such formulations, and methods for their administration to mammals, particularly humans.

The formulations contemplated by the present invention desirably comprise from about 0.2 to about 30% w/v of a substantially water-insoluble active pharmaceutical ingredient (API) and from about 1 to about 75% w/v of a water-miscible nonaqueous solvent into which the API is dissolved. The resulting formulation may, if desired, be water-free, although the inclusion of a pharmaceutically-acceptable aqueous solution therein is also contemplated. In the latter case, water may be present in the aqueous solution an amount ranging from about 0.2 to 98% w/v thereof, with the aqueous solution comprising water, saline and/or and other water-based solutions acceptable for administration to mammals.

In a preferred embodiment, the water-miscible nonaqueous solvent is an isosorbide, and more preferably DMI. The use of DMI permits the API to be dissolved therein and further permits the introduction of the aforementioned aqueous solution is desired. The amount of DMI included in the formulations may vary, but is desirably an amount sufficient to dissolve a therapeutic amount of the active pharmaceutical ingredient therein. Generally, this amount may range from about 50 to about 80% w/v of the formulation.

Preferred formulations of the invention, when used for parenteral administration, may include from about 0.1 to about 5 wt. % active pharmaceutical ingredient, from about 50 to about 80% w/v DMI, and from about 20 to about 50% w/v aqueous solution, wherein water desirably comprises from about 80 to about 100% w/v of the aqueous solution.

The formulation may desirably be packaged in a sterile vial, permitting administration to a mammal by injection, or in a sterile container for intravenous administration to the mammal. When an aqueous solution is included in the formulation, the formulation is desirably packaged in a sterile container which facilitates IV administration.

The formulations of the present invention may include many different classes of active pharmaceutical ingredients that heretofore were not satisfactorily prepared as parenterals due to the lack of a suitable solvent. Insoluble or poorly soluble compounds of marine or terrestrial origin may be solubilized using the present invention. Furthermore, these compounds may be of bacterial, fungal, plant or other natural origins.

An added benefit of the present invention arises from the ability of these APIs to be solvated in DMI. This permits a relatively lower amount of the API to be administered to a mammal in need, thereby reducing any potential side effects or toxicities that may be associated with the administration of relatively greater amounts of the API.

The present invention involves in part the discovery that dimethyl isosorbide is a versatile solvent capable of solubilizing a number of substantially water insoluble drugs in several drug classes. When formulated in accordance with the present invention, the formulations are stable, and well-suited for parenteral administration.

Substantially water-insoluble active pharmaceutical ingredients contemplated for use in the practice of the present invention include therapeutic agents, diagnostic agents, agents of nutritional value, and the like. Examples of therapeutic agents include: analgesics/antipyretics, anesthetics, antiasthmatics, antibiotics, antidepressants, antidiabetics, antifungal agents, antihypertensive agents, anti-inflammatories, antineoplastics, antianxiety agents, immunosuppressive agents, antimigraine agents, sedatives, antianginal agents, antipsychotic agents, antimanic agents, antiarrhythmics, antiarthritic agents, antigout agents, anticoagulants, thrombolytic agents, antifibrinolytic agents, hemorheologic agents, antiplatelet agents, anticonvulsants, antiparkinson agents, antihistamines/antipruritics, agents useful for calcium regulation, antibacterial agents, antiviral agents, antimicrobials, anti-infectives, bronchodialators, hormones, hypoglycemic agents, hypolipidemic agents, antiulcer/antireflux agents, antinauseants/antiemetics, oil-soluble vitamins (e.g., vitamins A, D, E, K, and the like).

Particularly preferred antineoplastic agents are the ansamycin derivatives, such as geldanmycin, including analogs (such as 17-allyl amino geldanmycin (17-AAG)) and derivatives thereof. Geldanmycin analogs and derivatives are well-known, with illustrative compounds described in U.S. Pat. Nos. 3,595,955, 4,261,989, 5,387,584, 5,932,566, 6,670,348, 6,887,993, 20030114450A1 and in International Published Applications WO09501342A1, WO9314215A1, WO9501342A1, WO03066005A2, WO00003737A2, WO0236574A and WO03066005A2, all of which are incorporated herein by reference.

Also preferred are microtubule stabilizing agents, such as the epothilones and analogs and derivatives thereof. Examples of epothilones contemplated by the present invention include, but are not limited to, epothilone A, epothilone B (EP0906), deoxyepothilone B, and epothilone B lactam (BMS-247550), and epothilone D. Another preferred microtubule stabilizing agent is discodermolide and analogs and derivatives thereof. Discodermolide analogs include, but are not limited to, 2-epi-discodermolide, 2-des-methyldiscodermolide, 5-hydroxymethyldiscodermolide, 19-des-aminocarbonyldiscodermolide, 9(13)-cyclodiscodermolide, and laulimalide.

Particularly preferred antibiotics are actinomycin and analogs and derivatives thereof, such as actinomycin A, C, C3 antibiotic complex, F1, F3, and Z complex.

Additional examples of relatively insoluble active pharmaceutical ingredients include those compounds which are substantially water insoluble and which are listed in the “Therapeutic Category and Biological Activity Index” of The Merck Index (12th Ed., 1996), the entire relevant contents of which are hereby incorporated by reference.

The formulations of the present invention may further include, if desired, one or more pharmaceutically-acceptable excipients. Illustrative of suitable excipients are solvents, carriers, diluents, disintegrants, and the like. While these excipients are well known in the art, examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil.

If desired, the inventive formulations may also include one or more lubricating agents, wetting agents, emulsifying and suspending agents, preserving agents, sweetening agents or flavoring agents. Examples of emulsifying agents include the Tweens, e.g., Tween 80 and related compounds, Cremophor and related compounds, tocopherol esters such as tocopheryl polyethylene glycol succinate and the like, Pluronics, emulsifiers based on polyoxy ethylene compounds, Span 80 and related compounds and other emulsifiers known in the art and approved for use in dosage forms suitable for administration to mammals (animals or humans).

The formulations may further be formulated to provide rapid, sustained or delayed release of the active ingredient after administration to the patient by employing procedures well known in the art.

The present invention further contemplates a method of preparing the aforesaid formulations. The method comprises the steps of dissolving the active pharmaceutical ingredient in dimethyl isosorbide, wherein the active pharmaceutical ingredient is selected from the group consisting of an ansamycin derived antineoplastic agent, an epothilone, actinomycin, an actinomycin analog, and combinations thereof.

Suitable active pharmaceutical ingredients; concentrations, etc., are as described above and set forth in the examples below.

The data summarized in Table I illustrate a few of the many different classes of substantially water insoluble drugs which may be solubilized in a water-miscible nonaqueous solvent such as dimethyl isosorbide. These and other substantially water-miscible drugs may now be satisfactorily prepared in parenteral formulations.

TABLE I

| | | Example Composition in Aqueous | | |
|---|---|---|---|---|
| | | Solution with DMI | | |
| | Solubility In | Conc. of Drug | Conc. of DMI | Conc. of Water |
| Drug Classes | DMI (mg/ml) | % (wt) | % (vol) | % (vol) |
| Taxanes and Epothilones | | | | |
| (Antineoplastic, | | | | |
| antimicrotubule agents) | | | | |
| Docetaxel | 90 | 3.38 | 75 | 25 |
| CY91 | 164 | 3.1 | 62 | 38 |
| CY75 | 166 | 2.6 | 53 | 47 |
| Rapamycin, Cyclosporine, | | | | |
| taxrolimusand analogs | | | | |
| (Immunosuppressive | | | | |
| agents) | | | | |
| Rapamycin | 121 | 5.86 | 48.3 | 51.7 |
| Rapamycin | | 1.38 | 55 | 45 |
| Rapamycin | | 0.45 | 45.5 | 54.5 |
| CY94 | 164 | | | |
| CY95 | 180 | 3.9 | 0.78 | 0.21 |
| Cyclosporine A | 311 | 21.5 | 69.2 | 30.8 |
| Cyclosporine A | | 2.88 | 57.6 | 42.4 |
| Cyclosporine A | | 1.43 | 57.3 | 42.7 |
| Cyclosporine A | | | | |
| Campothecins and | | | | |
| analogs (Antineoplastic, | | | | |
| Topoisomerase inhibitors) | | | | |
| Campothecin | <1 | | | |
| SN-38 | <1 | | | |
| CY1 | 52.2 | | | |
| CT19 | 95.8 | | | |
| CY30 | 27.8 | | | |
| CY57 | 14.3 | | | |
| CY3 | 23.0 | | | |
| CY55 | 179.2 | | | |
| CY59 | <1 | | | |
| CY4 | 8.4 | | | |
| Propofol and analogs | | | | |
| (anesthetic agents) | | | | |
| Propofol | completely | 30 | 60 | 10 |
| | miscible | | | |
| Propofol | | 7 | 65 | 28 |
| Propofol | | 1 | 54 | 45 |
| CY177 | 397 | | | |
| CY61 | 255 | | | |
| CY175 | 120 | | | |
| CY176 | 193 | | | |
| CY96 | 226 | | | |
| CY120 | 222 | | | |
| CT7 | 237 | | | |
| CY93 | 408 | | | |
| CY97 | 400 | | | |
| CT8 | 265 | 1.67 | 67 | 33 |
| CY155 | 242 | 3.5 | 70 | 30 |
| CY130 | 250 | 1.5 | 31 | 69 |
| CY135 | 160 | | | |
| Melatonin and analogs | | | | |
| Melatonin | 192 | 0.23 | 1.2 | >98.8 |
| CY9 | 170 | | | |
| CY14 | 246 | | | |
| CY15 | 274 | | | |
| CY17 | 194 | | | |
| CY19 | 160 | | | |
| CY49 | 278 | | | |
| Antineoplastic agents | | | | |
| Etoposide | 60 | | | |
| Etoposide | 60 | 5.9 | 52 | 48 |
| Etoposide | 40 | 4 | 24 | 76 |
| Etoposide | 20 | 2 | 73 | 27 |
| Antifungal, Antibacterial, | | | | |
| antiprotozoal, | | | | |
| antiinfective agents | | | | |
| Itraconazole | 9.6 | | | |
| Omeprazole | 17.4 | | | |

Those skilled in the art will recognize that variations are possible within the scope and spirit of this invention.

### EXAMPLE 1

### Preparation of Compositions

Drug compositions representative of the present invention were prepared by dissolving the desired drug in dimethyl isosorbide and/or water/or saline, or with gentle heating as needed. Other pharmaceutically suitable excipients could be added as needed. Table I contains specific amounts used in the various classes of drug compositions dissolved in DMI as exemplary ranges. Pharmaceutically acceptable dosage forms for parenteral administration were prepared by sterile filtration of the drug solutions and filling of vials under aseptic conditions.

### EXAMPLE 2

### Solubility of Taxanes in DMI and DMI-Water Mixtures

Taxane compositions were prepared according to Example 1. The solubility of taxotere and other taxane analogs ranged from about 90-166 mg/ml. The final pharmaceutical formulations were prepared either in neat DMI, or aqueous DMI from about 53-75% dimethyl isosorbide, and from about 25-47% water. Optionally, other pharmaceutically suitable excipients can be added as desired.

### EXAMPLE 3

### Solubility of Rapamycin and Analogs in DMI and DMI-Water Mixtures

Rapamycin compositions were prepared according to Example 1. The solubility of rapamycin and other rapamycin analogs ranged from about 121-180 mg/ml. The final pharmaceutical formulations were prepared either in neat DMI, or aqueous DMI from about 0.7-55% dimethyl isosorbide, and from about 0.2-55% water. Optionally, other pharmaceutically suitable excipients can be added as desired.

### EXAMPLE 4

### Solubility of Camptothecin and Analogs in DMI and DMI-Water Mixtures

Camptothecin compositions were prepared according to Example 1. The solubility of camptothecin and other camptothecin analogs ranged from about 1 -180 mg/ml., The final pharmaceutical formulation were prepared either in neat DMI, or aqueous DMI from about 1-60% dimethyl isosorbide, and from about 40-99% water. Optionally, other pharmaceutically suitable excipients can be added as desired.

### EXAMPLE 5

### Solubility of Propofol and Analogs in DMI and DMI-Water Mixtures

Propofol compositions were prepared according to Example 1. The solubility of propofol and other propofol analogs ranged from about 120-408 mg/ml. The final pharmaceutical formulation were prepared either in neat DMI, or aqueous DMI from about 31-70% dimethyl isosorbide, and from about 10-69% water. Optionally, other pharmaceutically suitable excipients can be added as desired.

### EXAMPLE 6

### Solubility of Melatonin and Analogs in DMI and DMI-Water Mixtures

Melatonin compositions were prepared according to Example 1. The solubility of melatonin and other melatonin analogs ranged from about 160-278 mg/ml. The final pharmaceutical formulations were prepared either in neat DMI, or aqueous DMI approximately 1.2-60% dimethyl isosorbide, and approximately 40-98% water. Optionally, other pharmaceutically suitable excipients can be added as desired.

### EXAMPLE 7

### Solubility of Other Poorly Water Soluble Drugs in DMI and DMI-Water Mixtures

Other compositions (see table I), e.g., for etoposide, itraconazole, and omeprazole were prepared according to Example 1. The solubility ranged from 1-60 mg/ml. The final pharmaceutical formulation were prepared either in neat DMI, or aqueous DMI from about 24-73% dimethyl isosorbide, and from about 27-76% water. Optionally, other pharmaceutically suitable excipients could be added as required, for e.g., surfactants.

What is claimed is:

1. A formulation for parenteral administration to a mammal comprising dimethyl isosorbide and a substantially insoluble active pharmaceutical ingredient selected from the group consisting of an ansamycin-derived antineoplastic agent, discodermolide, discodermolide analogs, an epothilone, actinomycin, an actinomycin analog, and combinations thereof.

2. The formulation of claim 1, wherein the active pharmaceutical ingredient is an ansamycin-derived antineoplastic agent.

3. The formulation of claim 2, wherein the ansamycin-derived antineoplastic agent is selected from the group consisting of geldanmycin, a geldanmycin derivative and a geldanmycin analog.

4. The formulation of claim 1, wherein the active pharmaceutical ingredient is an epothilone.

5. The formulation of claim 4, wherein the epothilone is selected from the group consisting of epothilone A, epothilone B (EPO906), deoxyepothilone B, epothilone B lactam (BMS-247550) and epothilone D.

6. The formulation of claim 1, wherein the active pharmaceutical ingredient is discodermolide.

7. The formulation of claim 1, wherein the active pharmaceutical ingredient is a discodermolide analog.

8. The formulation of claim 7, wherein the discodermolide analog is selected from the group consisting of 2-epi-discodermolide, 2-des-methyldiscodermolide, 5-hydroxymethyldiscoder-molide, 19-des-aminocarbonyldiscodermolide, 9(13)-cyclodiscodermolide, and laulimalide.

9. The formulation of claim 1, wherein the active pharmaceutical ingredient is an actinomycin analog.

10. The formulation of claim 9, wherein the actinomycin analog is selected from the group consisting of actinomycin A, C, C3 antibiotic complex, F1, F3, and Z complex.

11. The formulation of claim 1, wherein the formulation is nonaqueous.

12. The formulation of claim 1, the formulation further comprising a pharmaceutically-acceptable aqueous solution.

13. The formulation of claim 1, wherein dimethyl isosorbide is present in an amount of from about 0.2 to about 75% w/v of the composition.

14. The formulation of claim 13, wherein said dimethyl isosorbide is present in an amount of from about 0.2 to about 75% w/v of the composition and said composition comprises a pharmaceutically-acceptable aqueous solution in an amount of from about 0.2 to about 98% w/v.

15. The formulation of claim 2, wherein the formulation is nonaqueous.

16. The formulation of claim 2, the formulation further comprising a pharmaceutically-acceptable aqueous solution.

17. The formulation of claim 4, wherein the formulation is nonaqueous.

18. The formulation of claim 4, the formulation further comprising a pharmaceutically-acceptable aqueous solution.

19. The formulation of claim 6, wherein the formulation is nonaqueous.

20. The formulation of claim 6, the formulation further comprising a pharmaceutically-acceptable aqueous solution.

21. The formulation of claim 7, wherein the formulation is nonaqueous.

22. The formulation of claim 7, the formulation further comprising a pharmaceutically-acceptable aqueous solution.

23. The formulation of claim 9, wherein the formulation is nonaqueous.

24. The formulation of claim 9, the formulation further comprising a pharmaceutically-acceptable aqueous solution.

25. The formulation according to claim 14, wherein the formulation comprises about 0.1 to about 5 wt. % active pharmaceutical ingredient, about 50 to about 80% w/v DMI, and from about 20 to about 50% w/v aqueous solution.

26. A method of solubilizing a substantially water-insoluble active pharmaceutical ingredient comprising dissolving the active pharmaceutical ingredient in dimethyl isosorbide, wherein the active pharmaceutical ingredient is selected from the group consisting of an ansamycin-derived antineoplastic agent, discodermolide, a discodermolide analog, an epothilone, actinomycin, an actinomycin analog, and combinations thereof.

27. The method of claim 26, wherein the active pharmaceutical ingredient is an ansamycin-derived antineoplastic agent selected from the group consisting of geldanmycin, a geldanmycin derivative and a geldanmycin analog.

28. The method of claim 26, wherein the active pharmaceutical ingredient is an epothilone selected from the group consisting of epothilone A, epothilone B (EPO906), deoxyepothilone B, and epothilone B lactam (BMS-247550).

29. The method of claim 26, wherein the active pharmaceutical ingredient is a discodermolide analog selected from the group consisting of 2-epi-discodermolide, 2-des-methyldiscodermolide, 5-hydroxymethyldiscoder-molide, 19-des-aminocarbonyldiscodermolide, 9(13)-cyclodiscodermolide, and laulimalide.

30. The method of claim 26, wherein the active pharmaceutical ingredient is an actinomycin analog selected from the group consisting of actinomycin A, C, C3 antibiotic complex, F1, F3, and Z complex.

31. The method of claim 26, wherein the active pharmaceutical ingredient is discodermolide.

32. A method for administering a substantially water-insoluble active pharmaceutical ingredient to a mammal comprising preparing a formulation by dissolving an active pharmaceutical ingredient in dimethyl isosorbide and parenterally administering the resulting formulation to a mammal, wherein the active pharmaceutical ingredient is selected from the group consisting of an ansamycin-derived antineoplastic agent, discodermolide, a discodermolide analog, an epothilone, actinomycin, an actinomycin analog, and combinations thereof.

33. The method of claim 32, wherein the active pharmaceutical ingredient is an ansamycin-derived antineoplastic agent selected from the group consisting of geldanmycin, a geldanmycin derivative and a geldanmycin analog.

34. The method of claim 26, wherein the active pharmaceutical ingredient is an epothilone selected from the group consisting of epothilone A, epothilone B (EPO906), deoxyepothilone B, and epothilone B lactam (BMS-247550).

35. The method of claim 26, wherein the active pharmaceutical ingredient is a discodermolide analog selected from the group consisting of 2-epi-discodermolide, 2-des-methyldiscodermolide, 5-hydroxymethyldiscoder-molide, 19-des-aminocarbonyldiscodermolide, 9(13)-cyclodiscodermolide, and laulimalide.

36. The method of claim 26, wherein the active pharmaceutical ingredient is an actinomycin analog selected from the group consisting of actinomycin A, C, C3 antibiotic complex, F1, F3, and Z complex.

37. The method of claim 26, wherein the active pharmaceutical ingredient is discodermolide.
